(19) United States
(12) Patent Application Publication
(10) Pub. No.: US 2010/0286664 A1
(43) Pub. Date: Nov. 11, 2010

(54) CATHETER PUSH DEVICE

(75) Inventor: Thomas Haslinger, Sun City, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS, INC., Santa Clara, CA (US)

(21) Appl. No.: 12/463,002

(22) Filed: May 8, 2009

(51) Int. Cl. A61M 25/00

(52) U.S. Cl. 604/533

(57) ABSTRACT

A clamping device for gripping a catheter shaft to facilitate advancement in a patient's body lumen. The clamping device is preferably configured to be releasably secured to the catheter shaft and longitudinally slidable along the catheter shaft. The clamping device comprises a body portion that couples to a portion of the catheter adapter on a proximal end, and an axially directed internal lumen sized to receive the catheter shaft. Extending longitudinally from the body portion are opposed, resilient cantilevered fingers that flex inwardly against the catheter shaft upon application of digital pressure from the practitioner's thumb and forefinger to grip and capture the catheter shaft, thereby increasing the surface area of the interaction between the practitioner and the catheter and enhancing pushability.

### BACKGROUND OF THE INVENTION

This invention generally relates to intravascular catheters, such as balloon catheters used in percutaneous transluminal coronary angioplasty (PTCA) and stent delivery.

PTCA is a widely used procedure for the treatment of coronary heart disease. In this procedure, a balloon dilatation catheter is advanced into the patient's coronary artery and the balloon on the catheter is inflated within the stenotic region of the patient's artery to open up the arterial passageway and thereby increase the blood flow there through. To facilitate the advancement of the dilatation catheter into the patient's coronary artery, a guiding catheter having a pre-shaped distal tip is first percutaneously introduced into the cardiovascular system of a patient by the Seldinger technique through the brachial or femoral arteries.

The catheter is advanced until the pre-shaped distal tip of the guiding catheter is disposed within the aorta adjacent the ostium of the desired coronary artery, and the distal tip of the guiding catheter is then maneuvered into the ostium. A balloon dilatation catheter may then be advanced through the guiding catheter into the patient's coronary artery over a guidewire until the balloon on the catheter is disposed within the stenotic region of the patient's artery. The balloon is inflated to open up the arterial passageway and increase the blood flow through the artery. Generally, the inflated diameter of the balloon is approximately the same diameter as the native diameter of the body lumen being dilated so as to complete the dilatation but not over expand the artery wall. After the balloon is finally deflated, blood flow resumes through the dilated artery and the dilatation catheter can be removed therefrom.

In a large number of angioplasty procedures, there may be a restenosis, i.e. reformation of the arterial plaque. To reduce the restenosis rate and to strengthen the dilated area, physicians now frequently implant an intravascular prosthesis called a stent inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent is left in place within the artery at the site of the dilated lesion.

In both applications, the catheter must be advanced through the body to the heart. Control and advancement of catheters is difficult because of their construction. The user must frequently manipulate, or torque, the catheter shaft on the proximal end to facilitate advancement of the catheter with a desired orientation on the distal end. To provide the needed control over the movement of the catheter, it is necessary that these tubular catheters be made somewhat rigid. However, catheters must be flexible enough to navigate through the body lumen to arrive at the desired location within the body where the medical procedures will be performed. An overly rigid catheter shaft will not track, or follow, the guidewire. Therefore, reaching the desired location with a rigid catheter is more difficult. In some catheters the elongate tubular body is a hypotube formed of stainless steel, nitinol, or other suitable materials where, although the material is stronger, the diameter-to-length ratio is sufficiently low that the tubular body is flexible. This increases the capacity of the catheter to be advanced through the tortuous arterial passages of a patient and also improves pushability. Still, the catheter body is narrow with respect to a practitioner's hand/thumb/finger and thus it is often difficult to obtain and maintain a sure grip on the catheter shaft that allows for the necessary control, especially when various fluids are involved. Therefore, what has been needed is a device that improves torque ability of the catheter without interfering with the tracking and advancing of the catheter. The present invention satisfies these and other needs.

### SUMMARY OF THE INVENTION

The present invention is directed to a clamping member for gripping a catheter shaft to facilitate advancement on a patient's body lumen. The clamp is configured to be releasably compressed between a practitioner's thumb and forefinger to squeeze the catheter shaft. The clamp includes a cylindrical body portion that releasably attaches to the catheter proximal arm, a central lumen through which the catheter shaft is supported and translated, and resilient fingers extending from the body portion to flex against the catheter shaft upon digital pressure from the practitioner to frictionally grip and release the hypotube as needed for increased pushability.

The clamping member may be attached to a conventional catheter at the arm member via a thread on the nose piece. The clamping member can be threaded, push-fit, or otherwise coupled to the catheter arm at the initiation of the procedure. As the need for greater control over the distal portion of the catheter is needed, the practitioner detaches the clamping member from the catheter arm and advances it distally along the catheter body to the desired location. This may be close to the rotational hemostatic valve (“RHV”) in order to reduce the distance the practitioner must advance the clamping member, thereby increasing the usable force. The practitioner then grips the clamping member between the thumb and forefinger and squeezes the resilient fingers against the hypotube to frictionally grip the catheter body, increasing the surface area and control when compared to the bare catheter tube.

In one preferred embodiment of the present invention, the clamping member has a roughened surface on its inner opposing faces of the resilient fingers to effect an increased coefficient of friction when gripping the catheter body, which is useful if the catheter body has a lubricious or slick outer surface. An important aspect of this invention is not only the additional control it provides the user, but the added comfort the user experiences as the invention allows for more comfortable prolonged gripping. The user is thereby able to work with more control for a long period of time.

### BRIEF DESCRIPTION OF THE DRAWINGS

FIG. 1 is an elevational view of the catheter system embodying features of the invention, with a clamping device attached to an adapter.

FIG. 2 is an enlarged, elevated perspective view of the clamping member.

FIG. 3 is a cross-sectional view of the clamping member.

FIG. 4 is an elevational view of the catheter system with the clamping member displaced distally of the catheter arm into a position for gripping the catheter shaft.

### DETAILED DESCRIPTION OF THE INVENTION

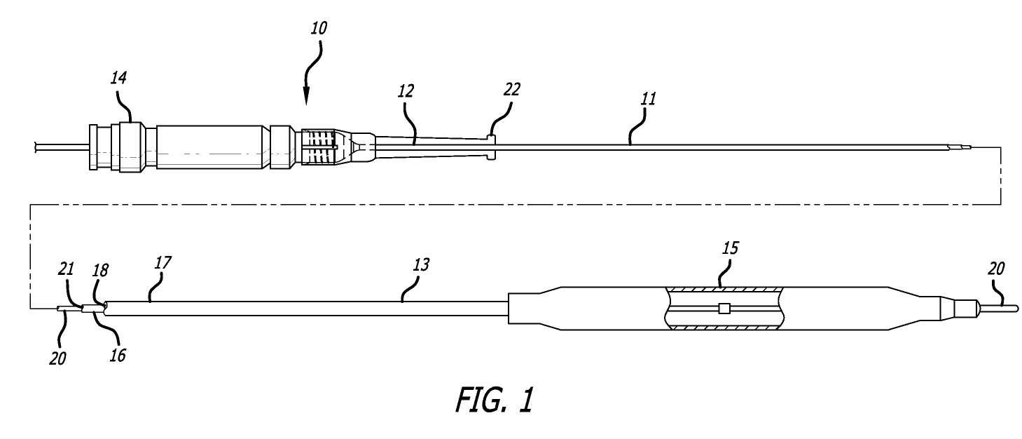

As shown in FIG. 1, the catheter 10 is an example of an over-the-wire catheter embodying features of the invention and generally includes a catheter shaft 11 having a proximal end 12, a distal end 13, an adapter 14, and a clamping member 22 slidably disposed about and capable of being releasably secured to the catheter shaft 11 at a location distal to an adapter 14. In the embodiment shown in FIG. 1, the clamping member 22 is detachably connected to the adapter 14. The catheter shaft 11 has an outer tubular member 17 and an inner tubular member 16 disposed within the outer tubular member 17 and defining, with the outer tubular member 17, an annular inflation lumen 18. Inflation lumen 18 is in fluid communication with an inflatable balloon 15. The outer tubular member may be constructed of a hypotube of Nitinol or other metal that, in the proper thickness to radial dimension ratio, provides a desirable pushability characteristics for the catheter shaft. An inflation fluid or gas is introduced into the inflation port (not shown) on the adapter 14, travels through the inflation lumen 18, and inflates the balloon 15. The inner tubular member 16 has an inner lumen 21 extending therein, which is configured to slidably receive a guidewire 20 suitable for advancement through a patient's coronary arteries. The distal extremity of the balloon 15 is sealingly secured to the distal extremity of the inner tubular member 16, and the proximal extremity of the balloon 15 is sealingly secured to the distal extremity of the outer tubular member 17. The construction of the balloon catheter is well known in the art, and further description of its construction and operation are omitted for brevity.

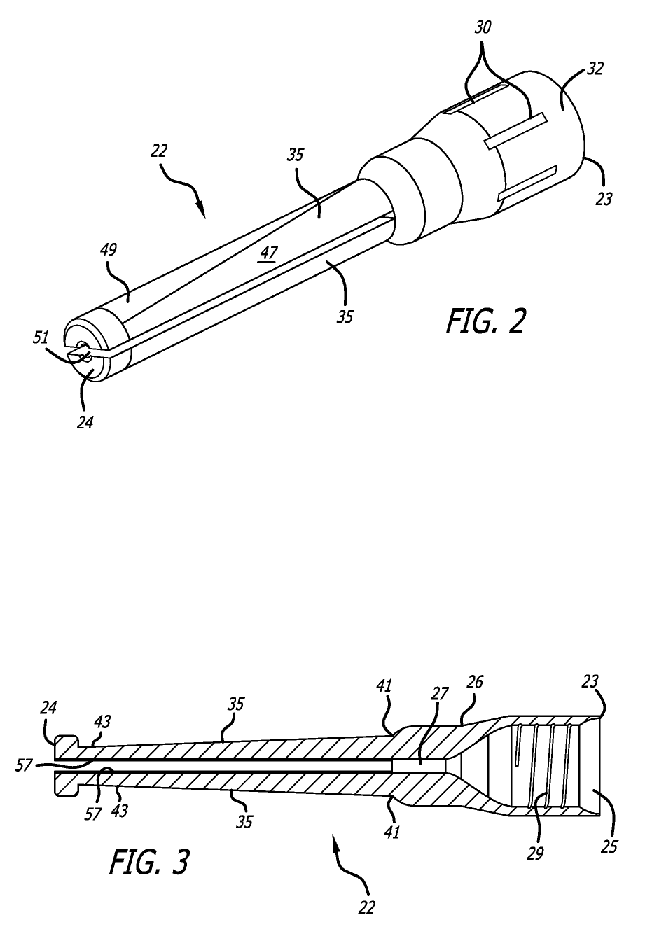

As shown in detail in FIGS. 2-4, the clamping member 22 has a proximal end 23 and a distal end 24. The proximal end 23 has a proximal port 25 having internal threads 29 for engaging mating external threads 31 on the outer surface of the catheter adapter arm member 33. Other coupling mechanisms are also envisioned for connecting the clamping member to a part of the catheter, such as snap-fit interlocking components, a quick release latching mechanism, and the like. In the stored position of FIG. 1, the clamping member 22 is coupled to the catheter adapter 14 via the internal threads 29 of the clamping member 22 and the external threads 31 of the adapter arm 33 to releasably lock the clamping member in a ready position for later use. The clamping member 22 has a lumen 27 extending from the proximal port 25 longitudinally through a cylindrical body portion 26 of the clamping member 22 and which receives and slides over the catheter shaft 11 with substantially no interference when the clamping member is not subjected to a radially inwardly directed force. The clamping member 22 in one embodiment is about 1 to about 2 inches long, and lumen 27 defines an inner diameter of, in the one embodiment, generally no less than about 0.040 inches (1.016 millimeters). The proximal port 25 of the clamping member 22 is sized and shaped to receive the distal portion of the arm member of the catheter adapter 33, such that the clamping member can be screwed onto and off of the adapter when needed. The clamping member preferably has a series of circumferentially spaced grooves 30 on an outer surface 32 to enhance the grip for the user when rotating the clamping member either onto or off of the adapter arm member.

The clamping member also includes a plurality of resilient fingers 35 extending in a cantilevered arrangement from the body portion 26 of the clamping member 22, such that the proximal ends 41 of the resilient fingers 35 are connected to the body portion 26 while the distal ends 43 of the resilient fingers 35 are free. In a preferred embodiment, the resilient fingers 35 are substantially semi-circular in cross-section and are integral with the body portion 26 as shown in FIG. 3. The outer surface 47 of the resilient fingers 35 many include a recessed or flattered area 49 for receiving the fingers of the user to promote more controlled pressure and to provide a convenient location for gripping and squeezing the clamping member 22. The resilient fingers may include a longitudinal recess 51 that is sized to receive a portion of the catheter shaft 11 such that the catheter shaft is partially captured by the clamping member 22 when pressure is applied to the outer surface 47 via the flattened areas 49.

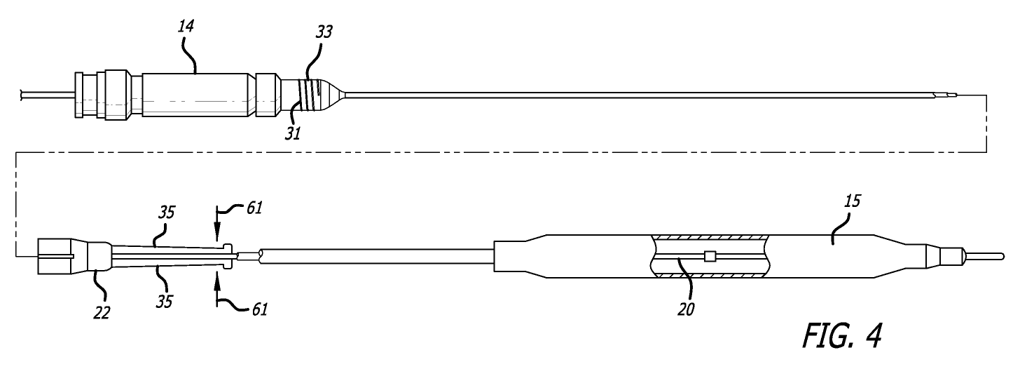

In use, the catheter is initially configured as shown in FIG. 1 with the clamping member 22 coupled via interlocking threads to the arm 33 of the catheter adapter 14. As the maneuverability of the catheter begins to decline, the practitioner can grip the outer surface 32 of the body portion 26 of the clamping member 22 and engage the circumferentially spaced grooves 30 to rotate the clamping member 22 with respect to the arm 33. The rotation of the clamping member 22 causes the clamping member to be released from the adapter 14, allowing the clamping member to slide in a distal direction along the catheter shaft 11 with the catheter shaft 11 disposed in the lumen 27 as well as between the resilient fingers 35. As shown in FIG. 4, the clamping member can be positioned distally to a location that reduces the length of catheter between the push point (i.e., the clamping member) and the balloon 15. Once the clamping member 22 is in place, the practitioner places a thumb and forefinger on respective sides of the resilient fingers against the flattened areas 49, and provides a squeezing (or radially inwardly directed) force as shown by arrows 61 to engage the catheter shaft 11 with the opposed inner surfaces of the respective resilient fingers 35. The inner surfaces 57 of the clamping member 22 can be knurled, roughened, or otherwise treated to enhance the frictional engagement of the resilient fingers with the outer surface of the catheter tubing. By squeezing the resilient fingers 35 against the catheter tubing 11, the resultant surface area engagement (i.e., the area of applied force between the clamping member 22 and the catheter tubing 11) is increased over the surface area engagement where the practitioner's fingers are used to squeeze the catheter tubing. Moreover, the wider surface area of the clamping member 22 provides a more comfortable and more efficient means by which the catheter can be grasped and manipulated, extending the duration by which the practitioner can comfortably perform the procedure. The catheter is then positioned within the arterial space as described in the background section of this application, where enhanced maneuverability is achieved by virtue of the present invention. When the correct position of the balloon 15 is obtained, the clamping device can be released, and the resilient fingers 35 are biased to return to the original, non-deformed position where it can be slid back to its original position and threaded onto the adapter 14. Should the clamping member be needed again for retracting the catheter, the same procedure can be applied to position and utilize the clamping device as needed.

Although individual features of embodiments of the invention may be shown in some of the drawings and not in others, those skilled in the art will recognize that individual features of one embodiment of the invention can be combined with any or all the features of another embodiment.

1. A clamping device for slidable translation over, and manual gripping of, an elongate catheter shaft of a dilation catheter comprising:
a body portion having a coupling mechanism for attaching the clamping device to an adapter at a proximal end of the catheter shaft, the body portion including a central lumen for receiving said elongate catheter shaft therein; and
first and second spaced apart, cantilevered, longitudinally extending fingers connected respectively at first ends to said body portion and aligned with said catheter shaft to position the catheter shaft therebetween, a spacing of the fingers permitting said clamping device to slidably translated over said catheter shaft;
wherein said cantilevered fingers are adapted to flex radially inward upon application of digital pressure to capture the catheter shaft therebetween.

2. The clamping device of claim 1 wherein the coupling mechanism comprises threads that engage mating threads on said adapter.

3. The clamping device of claim 1 wherein the first and second fingers further comprise respective axially extending, opposed channels adjacent the catheter shaft for partially capturing the catheter shaft upon application of the radially inward digital pressure.

4. The clamping device of claim 1 wherein the first and second fingers are substantially semi-circular and include respective recessed sections on an outer surface for providing a flat gripping surface.

5. The clamping device of claim 1 further comprising circumferentially spaced apart frictional grooves on said body portion.

6. The clamping device of claim 1 wherein the first and second fingers include a respective roughened surface on an opposed interior surface for increasing a frictional engagement with the catheter shaft.

7. The clamping device of claim 1 wherein the fingers are integral with the body portion.
